(19) United States
(12) Patent Application Publication
(10) Pub. No.: US 2012/0156137 A1
(43) Pub. Date: Jun. 21, 2012

(54) COMPOSITION FOR TREATMENT OF ATOPIC DERMATITIS COMPRISING GLUCOSAMINE AND DERIVATIVES THEREOF AND A METHOD FOR TREATMENT OF ATOPIC DERMATITIS USING THEM

(76) Inventors: Soo-youl Kim, Seocho-gu (KR); Chang-hoon Lee, Suwon-si (KR); Ai-young Lee, Jongno-gu (KR)

(21) Appl. No.: 12/446,238

(22) PCT Filed: Oct. 19, 2007
(86) PCT No.: PCT/KR2007/005156

§ 371 Date: May 9, 2011

(30) Foreign Application Priority Data
Oct. 20, 2006 (KR) 10-2006-0102461

(51) Int. Cl. A61K 31/7008; A61P 17/00; C12Q 1/52; C07H 5/06; A61K 49/00

(52) U.S. Cl. 424/9.2; 536/55.2; 514/62; 435/16

(57) ABSTRACT

Disclosed herein is a novel use of glucosamine or a derivative thereof. A pharmaceutical composition for treating atopic dermatitis comprising glucosamine or a derivative thereof and a method of treating atopic dermatitis using the composition are disclosed. The pharmaceutical composition and the method realize the safe treatment of atopic dermatitis without the side effects associated with conventional anti-inflammatory drugs.

### TECHNICAL FIELD

The present invention relates to a composition useful for the treatment of atopic dermatitis. More particularly, the present invention relates to a composition for treating atopic dermatitis comprising glucosamine or a derivative thereof and a method of treating atopic dermatitis using the composition.

### BACKGROUND ART

Atopic dermatitis is a skin disorder, the cause of which is unknown, which is typically characterized by intense itching, as well as dryness, redness and inflammation of the skin (Lancet. 1998; 351:1715-1721). Atopic dermatitis is a chronic disease that interferes with daily activities for a long period of time and most often affects infants and young children. It occurs in 10-15% of infants and young children. About 60% of atopic dermatitis patients exhibit signs of the disease in their first year, and about 85% develop the skin disease when they are equal to or less than five years of age (Lancet. 1998; 351:1715-1721). The condition gradually subsides as they grow in about 40% of cases of atopic dermatitis in children, but the prevalence of atopic dermatitis is increasing along with rapid industrialization, causing air pollution or environmental contamination, afflicting about 1-3% of adults, which is two to three times higher than before industrialization (Immunol. Allergy. Clin. North Am. 2002, 22:1-24; Lancet. 2003, 361:151-160). For these reasons, many researchers are preoccupied with efforts to reduce the clinical symptoms of atopic dermatitis.

The symptoms of atopic dermatitis differ from person to person. The main symptom is intense itching, followed by skin dryness, which gives patients an unpleasant feeling. The dryness damages the skin's protective surface, and renders the skin more susceptible to environmental irritants, to which the skin reacts, causing inflammation (Lancet. 1998, 351:1715-1721; J. Pediatr. Health Care. 2002, 16:143-145).

A variety of studies into the cause and treatment of atopic dermatitis have been made to date, but no theory for the accurate etiology and effective therapy of the disease has emerged, due to the complicated nature of the disease itself and conflicting research data. Currently available therapeutic agents include topical anti-inflammatory drugs, such as steroids (Br. J. Dermatol. 1999, 140:1114-1121), tacrolimus (J. Allergy Clin Immunol. 2002, 109:539-546; J. Am. Acad. Dermatol. 2001, 44:S58-S64), and pimecrolimus (Br. J. Dermatol. 2001, 144:788-794; J. Am. Acad. Dermatol. 2002, 46:495-504), antihistamines (Ann. Allergy Asthma Immunol. 1997, 79:197-211), and immunosuppressive drugs such as cyclosporine (Lancet. 1991, 338:137-140). As auxiliary therapies, low-irritant moisturizers or photochemotherapy using UVA (J. Am. Acad. Dermatol. 1998, 38:589-593; Lancet. 2001, 357:2012-2016) are clinically applied. However, since such therapies or therapeutic agents help reduce the symptoms to a tolerable level rather than provide fundamental treatment, they do not completely satisfy the demand of atopic dermatitis patients.

Topical steroids are most commonly used for treating atopic dermatitis. However, when administered for a long period of time, topical steroids cause Cushing's syndrome (Immunopharmacology. 1993, 25:269-276). Also, the sudden cessation of long-term administration of steroids causes acute adrenal insufficiency, which leads to general weakness, fever, myalgia, arthralgia, anorexia, etc., and brings about malfunction of the immune system, which increases the risk of bacterial or viral infection. In particular, the long-term application of steroids to the skin reduces the skin's protective ability, and the cessation of the use of steroids causes the severe onset of contact dermatitis. Thus, there is a need for drugs capable of treating atopic dermatitis with no such adverse effects.

Glucosamine is a natural amino sugar that has the molecular formula C₆H₁₃NO₆, and is classified as a hexosamine along with galactosamine since it has six carbon atoms. Glucosamine is a colorless needle-like crystalline powder that is degraded at 110° C., dissolves easily in water and is highly basic. Glucosamine is naturally present as a component of polysaccharides, which are widely distributed in chitin, bacterial cell walls and mucopolysaccharides, constituting animal cartilage and skin tissues. Glucosamine provides the raw material needed for recovering from osteoarthritis or injuries to joints. Specially, glucosamine plays a major role in the formation of a mu-copolysaccharide (called glycosaminoglycan) found in cartilage. It is also an important component in the structure of almost all body tissues including basal mucus membranes of the digestive and circulatory systems, synovial fluid, ligaments, and tendons of joints.

Glucosamine promotes mainly protein biosynthesis, stimulates the formation of a cartilage matrix, and stimulates the production of hyaluronic acid, which is an intra-articular lubricant. Due to such actions, glucosamine has been widely used in the treatment of primary osteoarthritis, rheumatoid arthritis, renal calculus and articular damage caused by accidents, and has been reported to be effective in the prevention of migraines (Russell, A. L. et al., Med. Hypotheses, 55(3):195-198 (2000)) and the treatment of chronic inflammatory bowel disease in children (Salvatore, S. et al., Aliment Pharmacol. Ther., 14:1567-1579 (2000)). However, there has been no report disclosing that glucosamine is effective in atopic dermatitis.

Transglutaminase is an enzyme that catalyzes the cross-linking of different proteins through the formation of ε-(γ-glutamyl) lysine bonds between a glutamine residue and a lysine residue (Methods in Enzymol. 1985, 113:358-375). The enzyme is considered to be an important factor in preventing skin damage and healing damaged tissues (FASEB J. 1999; 13: 1787-1795). However, abnormally activated transglutaminase contributes to the onset of several diseases, such as brain diseases, atherosclerosis, inflammatory diseases, autoimmune diseases and fibrosis (Neurochem. Int. 2002, 40:85-103).

Since transglutaminase 1 and 3 are abundantly expressed in the dermis of the skin, most studies involving transglutaminase in inflammatory skin diseases have been focused on transglutaminase 1 and 3 (J. Biol. Chem. 1999, 274:30715-30721). Transglutaminase 2 is involved in macrophage activity (Proc. Natl. Acad. Sci. USA. 2003, 100:7812-7817), and stimulates macrophages to release inflammatory cytokines, such as INF-γ and TNF-α. The function of transglutaminase 2 is applied to non-immune cells, such as fibroblasts and epidermal cells, and thus induces these cells to secrete inflammatory cytokines (J. Interferon & Cytokine Res. 2002, 22:677-682). The clinical pathological features of transglutaminase 2 are observed in the epidermal tissue of the small intestine of patients having celiac disease, which is an autoimmune disorder that damages the small intestine in response to gluten. The excess deposition of macrophages is seen in the epidermal tissue of the small intestine, and the activity of transglutaminase 2 is significantly increased in the macrophages (Prog. Exp. Tumor. Res. 2005, 38:158-73). In dermatitis herpetiformis, associated with celiac disease, like celiac disease, the excess deposition of macrophages is seen in the papillary dermis, where the activity of transglutaminase 2 is increased (J. Invest. Dermatol. 1999, 113:133-136).

In addition, a study using skin samples collected from atopic dermatitis and psoriasis patients revealed that macrophages are considerably activated at the dermoepidermal junction (Am. J. Dermatopathol. 1995, 17:139-144).

The present inventors have recently found that several synthetic peptides, which are transglutaminase inhibitors, have anti-inflammatory effects comparable to those of steroidal drugs when administered to a guinea pig model of pollen-induced allergic conjunctivitis (Sohn, J., Kim, T.-I., Yoon, Y.-H., and Kim, S.-Y. Transglutaminase inhibitor: A New Anti-inflammatory Approach in Allergic Coconjunctivitis. J. Clin. Invest. 111, 121-8, 2003; Soo-Youl Kim. Transglutaminase 2 in inflammation. Front Biosci. 11, 3026-3035, 2006). In addition, the present inventors, as described in Korean Pat. Application No. 10-2006-56942, conducted screening of natural substances already deemed safe and made commercially available in order to identify substances useful as transglutaminase inhibitors. The screening resulted in the finding that glucosamine or a derivative thereof has inhibitory activity toward transglutaminase.

### DISCLOSURE OF INVENTION

### Technical Problem

The inventors of this application conducted intensive and thorough research to investigate the speculation that inflammatory skin lesions exhibit high transglutaminase activity in atopic dermatitis and psoriasis patients. The research resulted in the finding that glucosamine or a derivative thereof is able to prevent the spread of inflammation in the skin of atopic dermatitis patients by inhibiting transglutaminase activity, thereby leading to the present invention.

### Technical Solution

It is therefore an object of the present invention to provide a pharmaceutical composition for treating atopic dermatitis comprising glucosamine, a derivative thereof, or a salt thereof.

It is another object of the present invention to provide a method of treating atopic dermatitis using glucosamine, a derivative thereof, or a salt thereof.

It is a further object of the present invention to provide a method of screening a candidate compound for the ability to inhibit transglutaminase activity in animals.

### BRIEF DESCRIPTION OF THE DRAWINGS

FIG. 1 is a graph showing the weight of tissue of ear biopsies from a 12-O-tetradecanoylphorbol 13-acetate (TPA)-induced mouse ear edema model, which was topically applied with glucosamine (Normal: no treatment; TPA-only: topically applied with TPA alone; G: topically applied with glucosamine; HC: topically applied with hydrocortisone);

FIG. 2 is a graph showing the results of a myeloperoxidase (MPO) assay of ear biopsies from a TPA-induced mouse ear edema model, which was topically applied with glucosamine;

FIG. 3 is a graph showing the weight of ear biopsies from a TPA-induced mouse ear edema model, which was orally administered with glucosamine (Normal: no treatment; Positive: a positive control; Vehicle: dosed with vehicle only; G: dosed with glucosamine (the number represents the dose of glucosamine); Dexamethasone: dosed with dexamethasone);

FIG. 4 is a graph showing the results of a MPO assay of ear biopsies from a TPA-induced mouse ear edema model, which was orally administered with glucosamine; and

FIG. 5 is a graph showing the results of a clinical test to determine the effects of glucosamine on atopy (blue bars: before glucosamine administration; yellow bars: one week after glucosamine administration; orange bars: two weeks after glucosamine administration), wherein data upon administration in a external use were displayed as a bar graph with an error range of less than 10%.

### BEST MODE FOR CARRYING OUT THE INVENTION

In one aspect, the present invention relates to a pharmaceutical composition for treating atopic dermatitis comprising glucosamine, a derivative thereof, or a salt thereof.

Glucosamine is a product of the degradation of chitosan, which is a major component of shells of crustaceans, such as crabs or shrimp. Chitin and chitosan are main components of crab or shrimp shells. Chitin is composed of 2-acetamido-2-deoxy-beta-D-glucose (N-acetylglucosamine). Chitosan is poly(beta-(1,4)-glucosamine), which is a polysaccharide derived through deacetylation from chitin. Glucosamine has the structure of Chemical Formula 1, below.

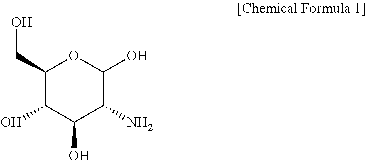

As used herein, the term “glucosamine derivative” refers to a compound in which a hydrogen of a hydroxyl group of glucosamine is replaced with an acyl or alkyl group, and has the structure of Chemical Formula 2, below.

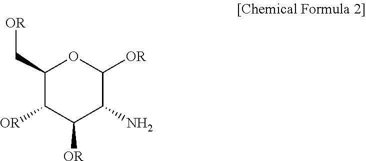

Wherein, R is an acyl group having 2 to 18 carbon atoms or a linear or branched alkyl group having 1 to 5 carbon atoms. Preferably, R may be an acyl group such as acetyl, propionyl, butyryl, pentanoyl, hexanoyl, heptanoyl, octanoyl, nonanoyl, decanoyl, undecanoyl, lauryl, tridecanoyl, myristyl, pentadecanoyl, palmitoyl, margaryl or stearyl, or may be an alkyl group such as methyl, ethyl, propyl, butyl, pentyl, isopropyl, isobutyl or secbutyl.

As described above, the introduction of hydrophobic groups into the alcohol groups of glucosamine at positions 1, 3, 4 and 6 without change in the amine group, which is believed to be responsible for the physiological activity of glucosamine. Thus, the resulting glucosamine derivative retains the innate physiological activity of glucosamine while being highly degradable in the environment and non-toxic and having high absorption capacity for heavy metals and antimicrobial activity.

Glucosamine suitable for use in the present invention may be obtained from any source of glucosamine. Glucosamine may be isolated and purified from a natural material, or may be commercially produced. Alternatively, glucosamine may be synthesized using any method suitable for the synthesis of pharmaceutically acceptable glucosamine. Alternatively, glucosamine may be obtained by isolating and purifying glucosamine from hydrolysates of chitin, hyaluronic acid, heparin and keratosulfate, including glucosamine or derivatives thereof, and other derivatives thereof.

The glucosamine or a derivative thereof, used in the present invention, may be included in the pharmaceutical composition of the present invention in the form of a pharmaceutically acceptable salt according to the intended use. Examples of such salt forms include sulfate, hydrochloride, acetate, citrate and malate of glucosamine or a glucosamine derivative, but are not limited thereto. A sulfate of glucosamine or a glucosamine derivative is preferred.

In addition, in order to maintain the stability of a pharmaceutically acceptable salt of glucosamine or a derivative thereof and to prevent the oxidation of an amino group of the salt form, the pharmaceutical composition may further include a known antioxidant, or the salt form may be bonded to a pharmaceutically acceptable metal salt, preferably a sodium or potassium salt.

Atopic dermatitis (also called atopic eczema) is a chronic recurrent eczematous disease. The fundamental cause and mechanisms giving rise to atopic dermatitis are not accurately understood. The disease seems to occur mainly due to unique genetic factors, but its cause is not simple, and multiple factors are implicated. Atopic dermatitis is a group of skin conditions that display clinical and histological progresses ranging from infant eczema to typical lichenification of dermatitis in children, adolescents and adults. The pharmaceutical composition of the present invention is effective in atopic dermatitis by preventing inflammatory reactions in the skin through the inhibition of transglutaminase by glucosamine, as an effective ingredient of the present composition, as described in the following Examples.

The pharmaceutical composition comprising glucosamine or a derivative thereof according to the present invention may be used singly or in combination with an approved pharmaceutical composition.

The pharmaceutical composition comprising glucosamine or a derivative thereof may be loaded into capsules in the absence of an excipient, or may be contacted uniformly and intimately with a finely divided solid carrier, a liquid carrier or both. Then, if necessary, the product is shaped into a desired form. Examples of suitable carrier vehicles include starch, water, saline, ethanol, glycerol, Ringer's solution, and dextrose solution. The pharmaceutical composition may be formulated into suitable dosage forms known in the art, according to Remington's Pharmaceutical Science, 19th Ed., 1995, Mack Publishing Company, Easton Pa.

The pharmaceutical composition comprising glucosamine or a derivative thereof according to the present invention is applicable in any formulation comprising glucosamine or a derivative thereof as an effective ingredient, and may be formulated into oral or parenteral forms. Preferred parenteral dosage forms are topically applied formulations, including ointments, emulsions, lotions and creams. An oral formulation is also preferable. For oral administration, the pharmaceutical composition of the present invention may be formulated, for example, into tablets, troches, lozenges, aqueous or oily suspensions, dispersible powders or granules, emulsions, hard or soft capsules, syrups or elixirs. Tablets and capsules may be formulated with a binder, such as lactose, saccharose, sorbitol, mannitol, starch, amylopectin, cellulose or gelatin, an excipient, such as dicalcium phosphate, a disintegrator, such as corn starch or sweet potato starch, and a lubricant, such as magnesium stearate, calcium stearate, sodium stearyl fumarate or polyethyleneglycol wax. When the present composition is formulated as capsules, a liquid carrier such as fatty oil may be further incorporated in the above materials.

For parenteral administration, the pharmaceutical composition of the present invention may be formulated, for example, in an injectable form for subcutaneous, intravenous or intramuscular injection, in a rectal suppository form, or in a spray form, such as an aerosol for nasal inhalation. An injectable dosage form may be formulated by mixing glucosamine or a glucosamine derivative with a stabilizer or a buffer in water to provide a solution or a suspension and packaging it in a unit-dose container, such as an ampule or a vial. The compound of the present invention may also be formulated in a rectal composition such as a suppository or a retention enema containing conventional suppository bases such as cocoa butter or other glycerides. For spray formulation, such as aerosol, the additives may be mixed with a propellant to disperse a water-dispersed concentrate or humidified powder. When the pharmaceutical composition is formulated as ointments, it may include oleaginous, water-soluble or emulsion ointment bases, antioxidants, antiseptics, moisturizers and solubilizers. The present pharmaceutical composition comprising glucosamine or a derivative thereof is preferably formulated into unit dosage forms through mixing under a preferred purity with a pharmaceutically acceptable carrier, that is, a carrier that is non-toxic to a recipient at a used concentration and dosage, and that can be combined with other formulation ingredients. In particular, the formulations preferably do not contain oxidants or other compounds known to be harmful to the body.

In another aspect, the present invention relates to a method of treating atopic dermatitis using glucosamine, a derivative thereof, or a salt thereof.

In one detailed aspect, the method comprises treating atopic dermatitis using the pharmaceutical composition comprising glucosamine, a derivative thereof, or a salt thereof.

The glucosamine, the derivative thereof, or the salt thereof according to the present invention may be administered as a pharmaceutical composition along with one or more pharmaceutically acceptable excipients. It will be apparent to those skilled in the art that when the pharmaceutical composition is administered to human patients, the suitable total daily dose may be determined by an attending physician within the scope of sound medical judgment. The specific therapeutically effective dose level for any particular patient may vary depending on a variety of factors, including the kind and degree of a desired reaction, the specific composition, including the use of any other agents according to the intended use, the patient's age, weight, general health, gender, and diet, the time of administration, route of administration, and rate of the excretion of the composition; the duration of the treatment, other drugs used in combination or coincidentally with the specific composition, and like factors well known in the medical arts. Suitable dosage forms known in the art are described in Remington's Pharmaceutical Science, 19th Ed., 1995, Mack Publishing Company, Easton Pa. Thus, the effective amount of the glucosamine or glucosamine derivative, suitable for the purposes of the present invention, is preferably determined taking into account the aforementioned factors.

In the present method, the glucosamine, the derivative thereof, or the salt thereof may be typically administered via various routes including oral and parenteral administration. Parenteral administration is preferred, and in this case, the compound is topically applied onto the area of the skin on which atopic dermatitis has developed.

In a preferred aspect, the present method may include administering glucosamine, a derivative thereof or a salt thereof, or a pharmaceutical composition comprising the same in combination with one or more known drugs for atopic dermatitis, such as anti-inflammatory drugs.

Pharmaceutically effective amounts of the drugs for atopic dermatitis are known in the art, and may be decided by an attending physician taking into account general conditions including the degree of symptoms and whether administration is to be in combination with glucosamine. The combined administration of glucosamine or a derivative thereof with a known drug for atopic dermatitis may alleviate side effects caused by the known drugs for atopic dermatitis, especially steroidal anti-inflammatory drugs, through the action of glucosamine or the derivative thereof, and may also result in additive or synergistic therapeutic effects. The known drugs for atopic dermatitis may be administered as a mixture with the glucosamine or the derivative thereof, or separately, either simultaneously or at different time points.

In a further aspect, the present invention provides a method of screening candidate compounds for ability to inhibit transglutaminase activity.

In one detailed aspect, the screening method comprises administering a candidate compound for inhibiting transglutaminase activity to an animal model in which an inflammatory reaction has been induced in the skin. Preferably, the candidate compound is topically applied or orally administered to the inflammation-induced animal model.

### MODE FOR THE INVENTION

A better understanding of the present invention may be obtained through the following examples which are set forth to illustrate, but are not to be construed as the limit of the present invention.

### Reference Example 1

### Preparation of TPA-Induced Mouse Ear Edema Model

Ear edema was induced in mice using 12-O-tetradecanoylphorbol 13-acetate (TPA), as follows. TPA was prepared at 2.5□/20□ in acetone. ICR mice were prepared one week before this test and allowed free access to food and water in order to minimize the stress of transport. 20□ of the TPA solution was applied onto right ears of male ICR mice (7 weeks old). After 24 hrs, mice were sacrificed by cervical dislocation, and the right ears were punched using a 6-mm biopsy punch. The ear punches were weighed using a microbalance, finely teased, and subjected to a myeloperoxidase (MPO) assay.

### Example 1

### Evaluation of Anti-Inflammatory Activity of Glucosamine Topically Applied to the TPA-Induced Mouse Ear Edema Model

Thirty male ICR mice (7 weeks old, weighing 25-32 g) were divided into six groups, each of which consisted of five mice. Experimental animals were allowed free access to food and water for one week in order to minimize the stress of transport. Glucos amine and a control drug were diluted in sterile distilled water and then diluted 1:9 in acetone.

Group 1 was not treated with TPA or any drug. Group 2 was treated with TPA alone on the right ear skin thereof. Group 3 was topically treated with TPA, and then with 20□ of 0.5% glucosamine in sterile distilled water/acetone (1:9) 15 min and 6 hrs after TPA application. According to the same procedure as in Group 3, Group 4 was topically treated with 1% glucosamine, and Group 5 with 2% glucosamine. As a positive control, Group 6 was topically treated with 0.5% hydrocortisone. 24 hrs after TPA application, mice were sacrificed by cervical dislocation, and ears were punched using a 6-mm biopsy punch. The ear punches were weighed and assayed for MPO activity.

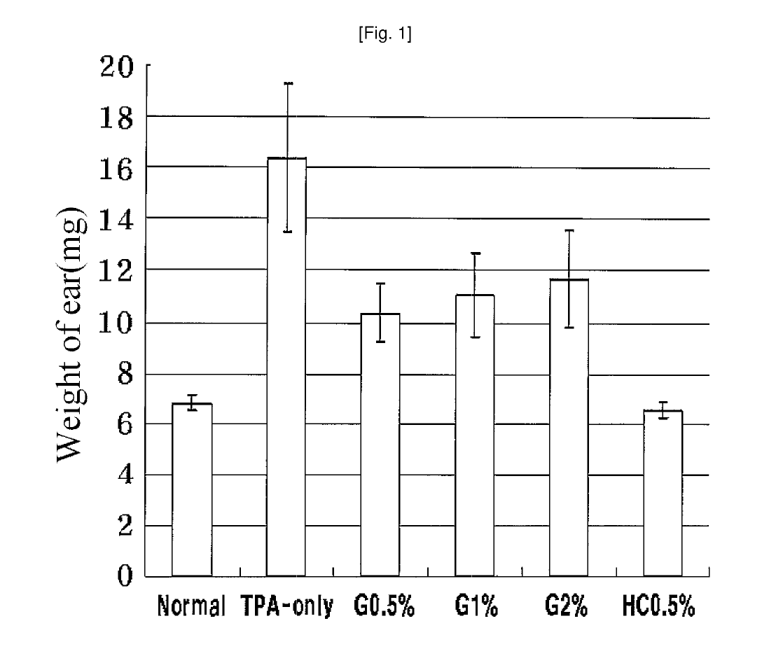

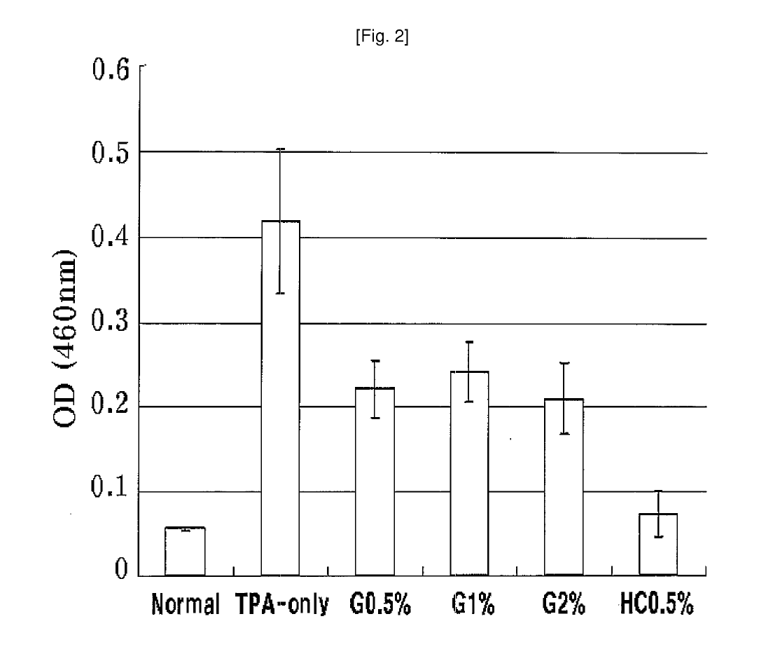

As shown in FIGS. 1 and 2, glucosamine was found to inhibit acute inflammation regardless of concentrations thereof.

### Example 2

### Evaluation of Anti-Inflammatory Activity of Glucosamine Orally Administered to the TPA-Induced Mouse Ear Edema Model

Thirty five male ICR mice (7 weeks old, weighing 25-32 g) were divided into seven groups, each of which consisted of five mice. Experimental animals were allowed free access to food and water for one week in order to minimize the stress of transport. Glucosamine and a control drug were diluted in sterile physiological saline.

Group 1 was not treated with TPA or any drug. Group 2 was treated with TPA alone on the right ear skin thereof. Group 3 was topically applied with TPA, and then orally administered through a sonde with 0.5 ml of glucosamine in sterile physiological saline 15 min and 6 hrs after TPA application. According to the same procedure as in Group 3, Group 4 was orally administered with 0.5 ml of glucosamine (5 mg/kg), Group 5 with 0.5 ml of glucosamine (10 mg/kg), Group 6 with 0.5 ml of glucosamine (20 mg/kg), and Group 7 with 0.5 ml of dexamethasone (10 mg/kg). 24 hrs after TPA application, mice were sacrificed by cervical dislocation, and ears were punched using a 6-mm biopsy punch. The ear punches were weighed and assayed for MPO activity.

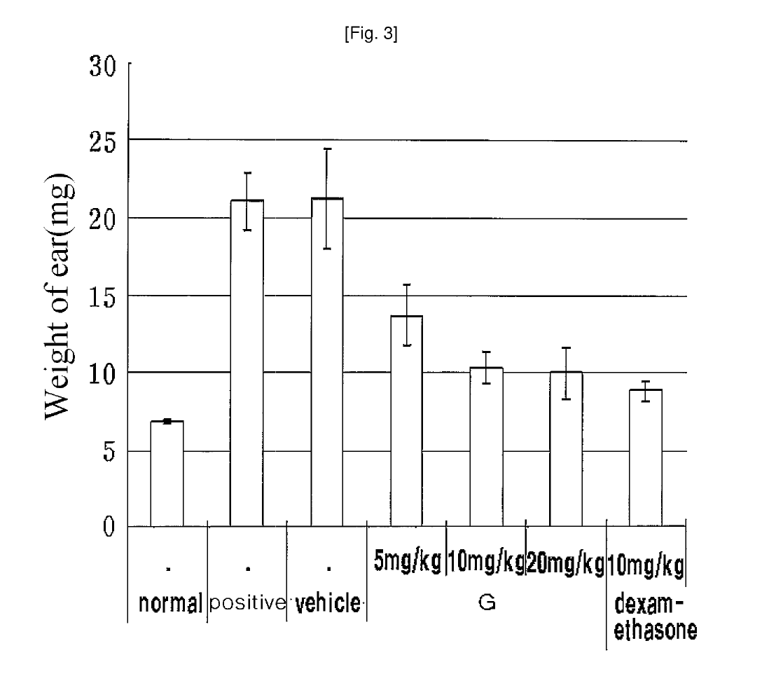

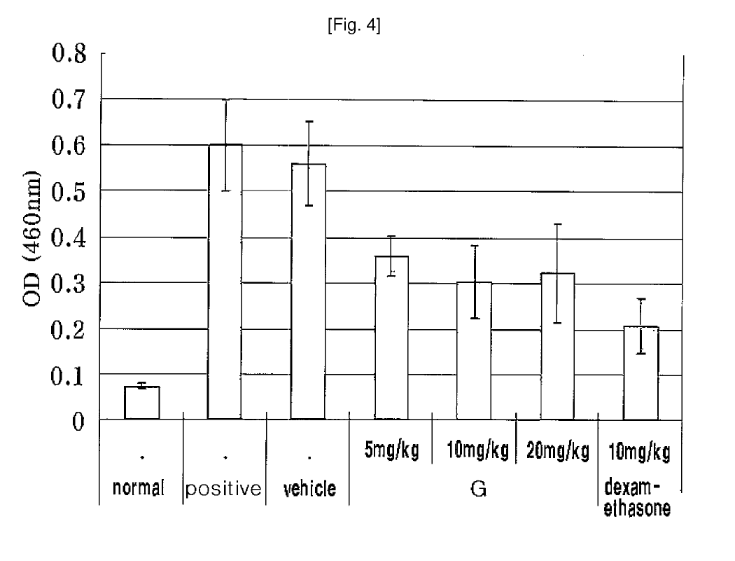

As shown in FIGS. 3 and 4, when orally administered, glucosamine inhibited acute inflammation regardless of the concentration thereof.

### Example 3

### Clinical Test for Therapeutic Effects of Glucosamine on Atopy

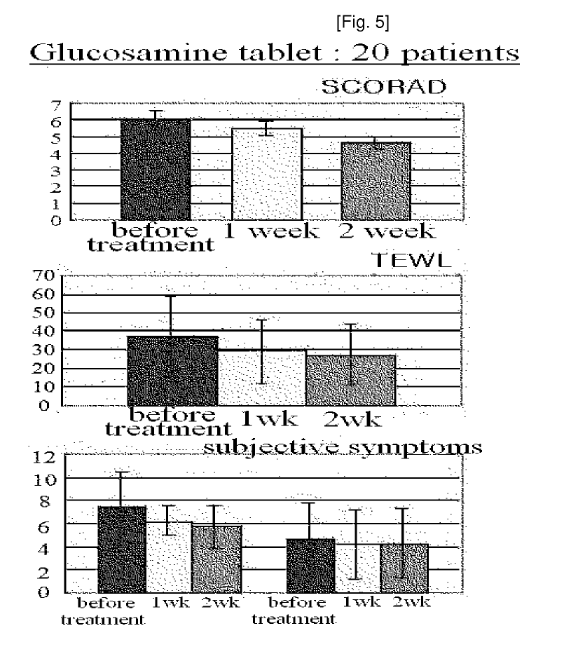

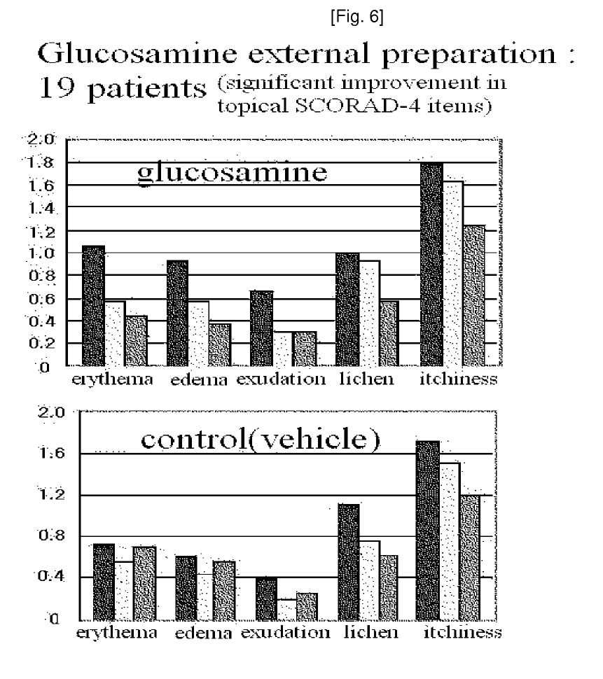

A clinical test was performed with twenty atopy patients in order to determine whether glucosamine has therapeutic effects on atopy in practice. The clinical test was conducted by the Department of Dermatology, Dongguk University Hospital (attending physician: A Y Lee; the clinical test was approved by an IRB of Dongguk University Hospital). Twenty atopy patients were dosed with glucosamine in the form of a tablet and a cream. In brief, glucosamine was orally administered at a daily dose of 500 mg for a period of two weeks. The cream (5% glucosamine) was topically applied to a desired site of the skin twice a day for a period of four weeks. The results are shown in FIG. 5. As shown in FIG. 5, glucosamine exhibited significant therapeutic effects on atopy in both oral administration and external use. These results indicate that glucosamine has therapeutic effects on atopy upon both oral and parenteral administration thereof.

### INDUSTRIAL APPLICABILITY

As described hereinbefore, glucosamine, a derivative thereof, or a salt thereof according to the present invention has good therapeutic effects on atopic dermatitis. The compound is safe, avoiding the side effects encountered in conventional steroidal drugs, and thus has potential as an anti-inflammatory drug.

1. A pharmaceutical composition for treating atopic dermatitis comprising glucosamine, a derivative thereof, or a salt thereof.

2. The pharmaceutical composition according to claim 1, wherein the glucosamine derivative is represented by the following Chemical Formula 2: 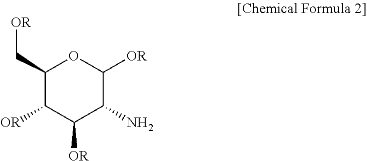
wherein R is an acyl group having 2 to 18 carbon atoms or a linear or branched alkyl group having 1 to 5 carbon atoms.

3. A method of treating atopic dermatitis comprising administering glucosamine, a derivative thereof, or a salt thereof.

4. The method according to claim 3, wherein the glucosamine derivative is represented by the following Chemical Formula 2: 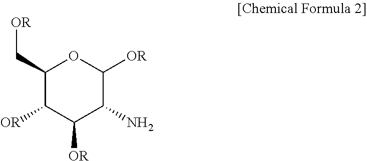
wherein R is an acyl group having 2 to 18 carbon atoms or a linear or branched alkyl group having 1 to 5 carbon atoms.

5. The pharmaceutical composition according to claim 1, wherein the salt of the glucosamine or the derivative thereof is a sulfate.

6. The method according to claim 3, wherein the salt of the glucosamine or the derivative thereof is a sulfate.

7. A method of screening a transglutaminase inhibitor comprising administering a candidate compound for inhibiting transglutaminase activity to an animal model in which an inflammatory reaction is induced in the skin.
